(19) United States
(12) Patent Application Publication
(10) Pub. No.: US 2013/0152951 A1
(43) Pub. Date: Jun. 20, 2013

(54) Intravenous Site Shield

(71) Applicant: Joseph R. Nigro, Covina, CA (US)

(72) Inventor: Joseph R. Nigro, Covina, CA (US)

(21) Appl. No.: 13/331,232

(22) Filed: Dec. 20, 2011

(51) Int. Cl. A61F 13/10

(52) U.S. Cl. 128/888

(57) ABSTRACT

A shield seals out moisture over a portion of a wearer's limb. The shield may be used to protect chronic Intravenous (IV) sites preventing moisture from reaching the IV site and provides a moisture tight compartment for carrying personal items during aquatic activities. The shield is made of a thermoplastic elastomer material which freely stretches and includes two opposing very small entry openings which stretch to form collar regions at each stretched opening providing an area versus a line of contact between the shield and skin thereby sealing out moisture. The improved sealing of the present invention allows many activities previously denied to patients. The shield is preferably generally spherical but may be elliptical, football shaped, oval, or other shapes and may have one closed end or multiple openings for fingers or toes.

### BACKGROUND OF THE INVENTION

The present invention relates to intravenous sites and in particular to shielding chronic intravenous sites from contamination due to common physical activities.

Medical patients often require medication and/or nutrients to be administered via an Intravenous (IV) device. Usually, an IV catheter is inserted into a vein in the patient's forearm or leg and then connected to an IV bag containing an IV fluid of the medication and/or nutrients through catheter tubing, IV tubing. After the administration of the IV fluid, the IV catheter may be removed or left in place for future administration of IV fluids. Due to an increasing tendency to reduce hospital stay length (and therefore costs) a patient requiring future administration of IV fluids may be sent home with the IV catheter remaining implanted in the patient's vein. After returning home, when the patient is in need of the IV fluids, a nurse can remove a plug and luer, and then attach IV tubing and an IV bag to the IV site.

Unfortunately, any moisture collecting around the IV site may act as a medium for the entry of bacteria into the vein through the break in the skin where the IV catheter is inserted. As a result, an IV site must be protected from exposure to moisture, and, for example, showering is not permitted.

Various attempts have been made to design a device for protecting such IV sites to allow normal washing, showering, and the like, but no device has proven practical and effective.

### BRIEF SUMMARY OF THE INVENTION

The present invention addresses the above and other needs by providing a shield which seals out moisture over a portion of a wearer's limb. The shield may be used to protect chronic Intravenous (IV) sites preventing moisture from reaching the IV site and provides a moisture tight compartment for carrying personal items during aquatic activities. The shield is made of a thermoplastic elastomer material which freely stretches and includes two opposing very small entry openings which stretch to form collar regions at each stretched opening providing an area versus a line of contact between the shield and skin thereby sealing out moisture. The improved sealing of the present invention allows many activities previously denied to patients. The shield is preferably generally spherical but may be elliptical, football shaped, oval, or other shapes and may have one closed end or multiple openings for fingers or toes.

In accordance with one aspect of the invention, there is provided a shield for chronic Intravenous (IV) sites. The shield comprising a body having a diameter between three and twelve inches and at least one opening in the body having a relaxed diameter of less than two inches, wherein the opening is stretchable to fit over a patient's limb and form a moisture proof seal. The body is preferably a thermoplastic elastomer material having an elongations of at least 800 percent, a hardness of Shore A 8+/−5, and a tensile strength of between 800 and 1,200 PSI. An example of a suitable thermoplastic elastomer material is composed of Styrene-Ethylene/Butylene-Styrene (SEBS), a CoPolymer, Anti-Oxidants, and UPS oil. A preferred embodiment of the thermoplastic elastomer material comprises between ten and twenty percent SEBS and between eighty and ninety percent USP oil.

### BRIEF DESCRIPTION OF THE SEVERAL VIEWS OF THE DRAWING

The above and other aspects, features and advantages of the present invention will be more apparent from the following more particular description thereof, presented in conjunction with the following drawings wherein:

FIG. 1 shows a typical Intravenous (IV) site.

FIG. 2 shows an IV site with an IV tube removed and a shield according to the present invention protecting the IV site from moisture.

FIG. 3A is a side view of the shield according to the present invention in a relaxed state.

FIG. 3B is an end view of the shield according to the present invention in the relaxed state.

FIG. 4A is a side view of the shield according to the present invention in a stretched state.

FIG. 4B is an end view of the shield according to the present invention in the stretched state.

FIG. 5A is a side view of an elongated shield according to the present invention in a relaxed state.

FIG. 5B is an end view of the elongated shield according to the present invention in the relaxed state.

FIG. 6A is a side view of the elongated shield according to the present invention in a stretched state.

FIG. 6B is an end view of the elongated shield according to the present invention in the stretched state.

FIG. 7A is a side view of a closed end shield according to the present invention in a relaxed state.

FIG. 7B is an end view of the closed end shield according to the present invention in the relaxed state.

FIG. 8A is a side view of a multi-opening shield according to the present invention in a relaxed state.

FIG. 8B is an end view of the multi-opening shield according to the present invention in the relaxed state.

Corresponding reference characters indicate corresponding components throughout the several views of the drawings.

### DETAILED DESCRIPTION OF THE INVENTION

The following description is of the best mode presently contemplated for carrying out the invention. This description is not to be taken in a limiting sense, but is made merely for the purpose of describing one or more preferred embodiments of the invention. The scope of the invention should be determined with reference to the claims.

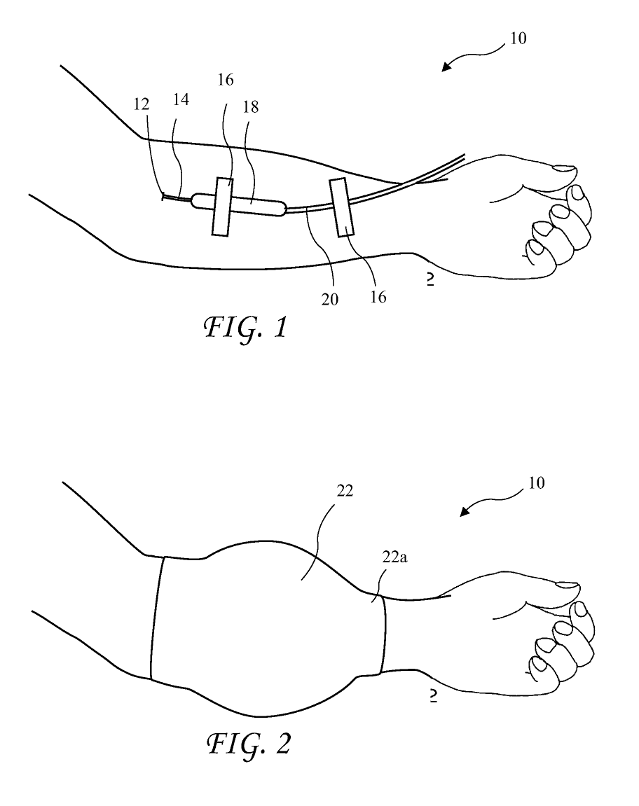

A typical Intravenous (IV) site 12 is shown in FIG. 1. A catheter 18 includes a Peripherally Inserted Central Catheter (PPIC) (or alternatively a needle) 14 piercing a patients arm 10 at the IV site 12. The catheter 18 is held in place by tape 16. An IV tube 20 is connected to the catheter 18 to carry IV fluids to the patient. When unprotected, moisture around the IV site 12 may carry bacteria into a vein infecting the patient.

The IV site 12 with a shield 22 according to the present invention protecting the IV site 12 from moisture is shown in FIG. 2. The IV tube 20 has been removed and the shield 22 prevents moisture from reaching the catheter 18 which remains in place. The shield 22 is made from an engineered material having elastic properties causing the shield 22 to form collars 22a and 22b at entry and exit points into the shield 22. The collars 22a and 22b provide an area of contact with the patient's arm (or leg) and thus provide a significantly improved seal compared to know IV site shields which only provide a line of contact, not an area.

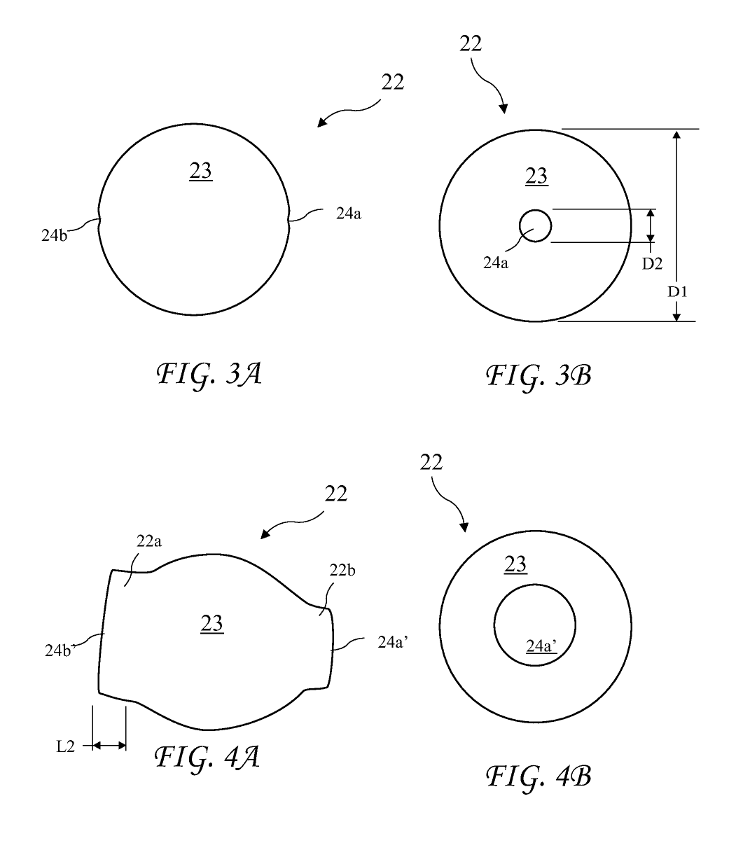

A side view of the shield 22 in a relaxed state is shown in FIG. 3A and an end view of the shield 22 in the relaxed state is shown in FIG. 3B The relaxed shield is approximately spherical and has a diameter D1 of preferably between three and twelve inches and two small relaxed openings 24a and 24b. The relaxed openings 24a and 24b have diameters D2 of preferably less than two inched, more preferably less that 1.5 inches, and most preferably approximately 0.5 inches. The relaxed shield body 23 is preferably generally spherical but may be elliptical, football shaped, oval, or other shapes.

A side view of the shield 22 in a stretched state is shown in FIG. 4A and an end view of the shield 22 in the stretched state is shown. The entry and exit 24a and 24b are shown stretched to fit over a patient's limb to form collars 22a and 22b. The collars 22a and 22b are a length L2 which extends over the patient's limb forming an area not just a line of contact and as a result, a moisture tight seal.

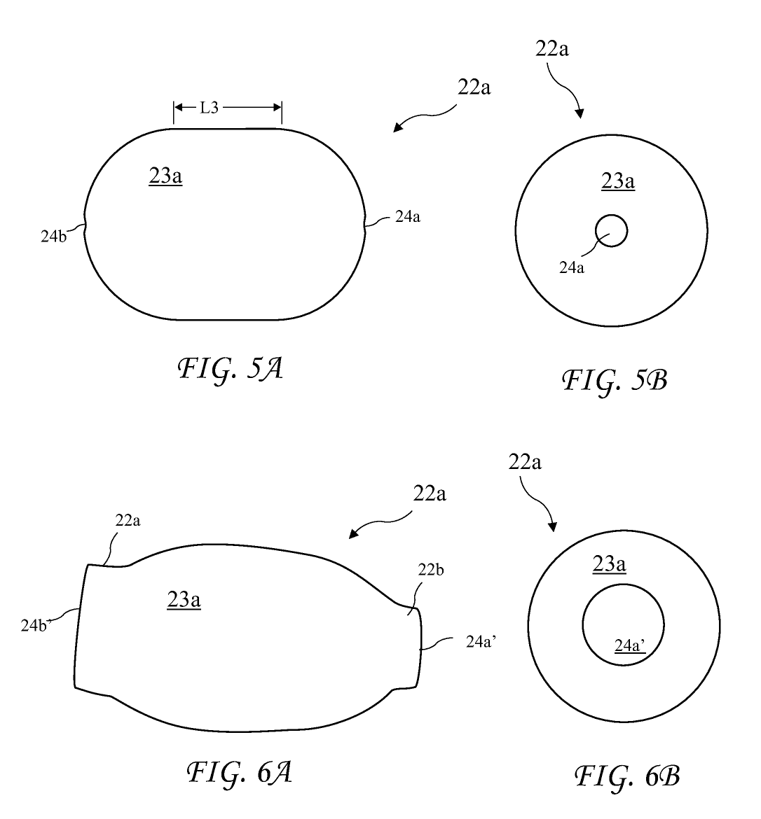

A side view of an elongated shield 22a according to the present invention in a relaxed state is shown in FIG. 5A, an end view of the elongated shield 22a is shown in FIG. 5B, a side view of the elongated shield 22a in a stretched state is shown in FIG. 6A, and an end view of the elongated shield 22a in the stretched state. The elongated shield 22a is of an oval cross-section and allows the elongated shield 22a to protect a longer portion of a patient's limb than a spherical shield, but is otherwise similar to the shield 22.

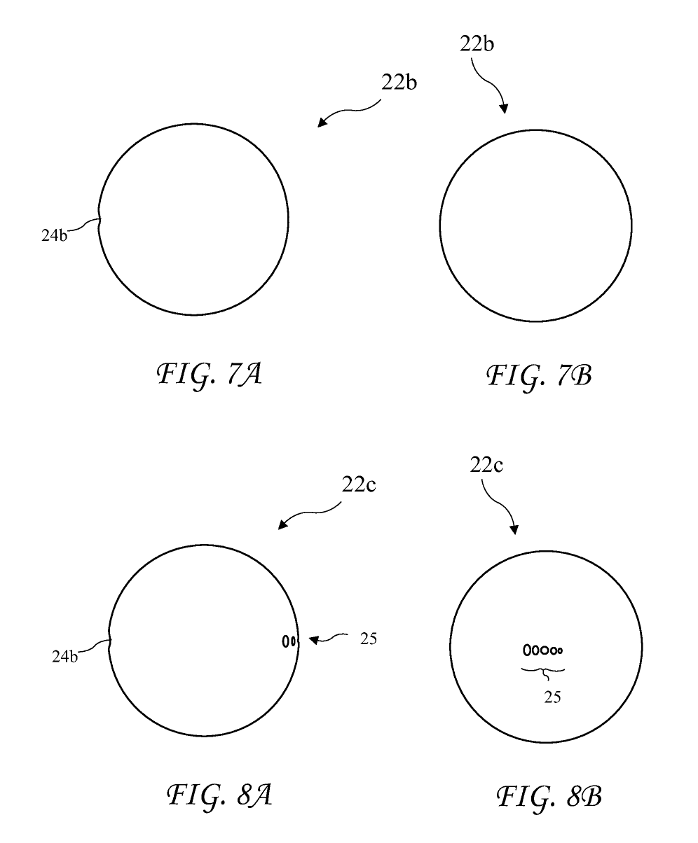

Aside view of a closed end shield 22b according to the present invention, is shown in a relaxed state FIG. 7A and an end view of the closed end shield 22b is shown in the relaxed state FIG. 7B. The closed end shield 22b may be used over an hand or foot to for a moisture proof seal, or over the end of a limb following an amputation, but is otherwise similar to the shield 22.

A side view of a multi-opening shield 22c according to the present invention is shown in a relaxed state in FIG. 8A and an end view of the multi-opening shield 22c is shown in the relaxed state in FIG. 8B. The multi-opening shield 22c allows the shield to reside over a hand or foot with fingers or toes exposed, but is otherwise similar to the shield 22.

The shield body 23 is made of an engineered thermoplastic elastomer having a tensile strength of between 800 and 1200 PSI, a hardness of about Shore 8+/−5, and a minimum elongation of 800 percent and a preferred elongation between 1200 and 1500 percent. An example of a suitable thermoplastic elastomer is composed of Styrene-Ethylene/Butylene-Styrene (SEBS), a CoPolymer, Anti-Oxidants, and UPS oil, and a preferred embodiment includes between ten and twenty percent SEBS and between eighty and ninety percent USP oil.

While the invention herein disclosed has been described by means of specific embodiments and applications thereof, numerous modifications and variations could be made thereto by those skilled in the art without departing from the scope of the invention set forth in the claims.

I claim:

1. A shield for chronic Intravenous (IV) site, the shield comprising:
a body made from a very high elongation material;
at least one opening in the body having relaxed diameter less than two inches; and
the entry opening stretchable to fit over a patient's limb and form a moisture proof seal.

2. The shield of claim 1, wherein the very high elongation material has a minimum elongation of 800 percent.

3. The shield of claim 2, wherein the very high elongation material has a minimum elongation of 1200 percent.

4. The shield of claim 3, wherein the very high elongation material has an elongation of about 1,500 percent.

5. The shield of claim 1, wherein the at least one opening has a relaxed diameter of less than 1.5 inches.

6. The shield of claim 5, wherein the at least one opening has a relaxed diameter of about 0.5 inches.

7. The shield of claim 1, wherein the at least one opening comprises two openings on opposite sides of the shield.

9. The shield of claim 1, wherein the at least one opening comprises a first opening having a diameter less than two inches and five smaller second opening opposite the first opening configured to receive five fingers of a patient.

10. The shield of claim 1, wherein the shield has a generally spherical relaxed shape.

11. The shield of claim 1, wherein the shield has a generally elliptical relaxed shape.

12. The shield of claim 1, wherein the shield has a generally oval relaxed shape.

13. The shield of claim 1, wherein the shield is made from a thermoplastic elastomer material.

14. The shield of claim 13, wherein the thermoplastic elastomer material has a hardness of Shore A 8+/−5.

15. The shield of claim 14, wherein the thermoplastic elastomer material has a tensile strength of between 800 and 1,200 PSI.

16. The shield of claim 5, wherein the thermoplastic elastomer material consists essentially of Styrene-Ethylene/Butylene-Styrene, a CoPolymer, Anti-Oxidants, and UPS oil.

17. A shield for chronic Intravenous (IV) site, the shield comprising:
a body made from a material having an elongations of at least 800 percent;
at least one opening in the body having relaxed diameter less than two inches; and
the opening stretchable to fit over a patient's limb and form a moisture proof seal.

18. The shield of claim 17, wherein the thermoplastic elastomer material consists essentially of Styrene-Ethylene/Butylene-Styrene, a CoPolymer, Anti-Oxidants, and UPS oil.

19. A shield for chronic Intravenous (IV) site, the shield comprising:
a body comprising:
a thermoplastic elastomer material;
a diameter between three and twelve inches;
an elongations of at least 800 percent;
a hardness of Shore A 8+/−5; and
a tensile strength of between 800 and 1,200 PSI;
at least one opening in the body having relaxed diameter of less than two inches; and
the opening stretchable to fit over a patient's limb and form a moisture proof seal.

20. The shield of claim 19, wherein the thermoplastic elastomer material comprises between ten and twenty percent SEBS and between eighty and ninety percent USP oil.
